(19) United States
(12) Patent Application Publication
(10) Pub. No.: US 2025/0377306 A1
(43) Pub. Date: Dec. 11, 2025

(54) REDUCTION OF STRAY LIGHT NOISE IN OPTICAL RAMAN PROBE SENSORS

(71) Applicant: Merck Patent GMBH, Darmstadt (DE)

(72) Inventors: Laurent Jourdainne, Stutzheim-Offenhein (FR); Celia Sanchez, Grenoble (FR); Celine Duchemin, Montbonnot Saint Martin (FR); Renaud Puget, Bernin (FR); Fabrice Thomas, Saint Martin D'Heres (FR)

(21) Appl. No.: 18/874,501

(22) PCT Filed: Aug. 4, 2023
(86) PCT No.: PCT/EP2023/071670

§ 371 Date: Dec. 12, 2024

(30) Foreign Application Priority Data
Aug. 2, 2022 (EP) 22315179.6

(51) Int. Cl. G01N 21/65; C12M 1/00; C12M 1/34

(52) U.S. Cl. CPC G01N 21/65; C12M 23/26; C12M 41/30; G01N 2201/0636

(57) ABSTRACT

A cap having a closed end and one or more openings is attached to the tip of an optical Raman probe sensor. The cap serves to block stray light noise from entering the tip of the sensor. In this way, Raman spectra may be more accurate and consistent. Further, the cap may be permanently affixed or removably attached to the sensor. In some embodiments, a reflective surface may be included on the interior surface of the closed end of the cap. This reflective surface may reflect Raman scattering light toward the tip, enhancing the received signal by a factor of 2 to 100.

### FIELD

Embodiments of the present disclosure relate to systems and method to reduce stray light noise in optical Raman probe sensors.

### BACKGROUND

In many applications, such as bioprocessing applications, it is important to carefully and accurately monitor the composition of materials. For example, in a bioreactor, it may be important to monitor the amounts of various molecules, such as glucose, lactate, glutamine, ammonium and others.

In many situations, this monitoring may be done using Raman spectroscopy. In Raman spectroscopy, a laser is used to direct light at a specific wavelength toward a target molecule. A photon reaches a molecule and excites it. Once the photon excites the molecule, there are several possible results. The most common is that the excitation is temporary and the molecule returns to its initial energy state. In this mode, the photon is scattered or redirected due to the interaction with the molecule. Further, the wavelength of the photon is unchanged since none of its energy was absorbed by the molecule. This phenomenon is referred to as Rayleigh scattering and does not provide any information about the molecule being analyzed.

In another mode, the molecule is excited by the photon and moves to a different vibrational or rotational state. If that new state is a higher energy state than the original energy state, then the photon loses energy, which results in a lower frequency. In this way, the total amount of energy is conserved. This mode is referred to as Stokes Raman scattering.

If that new state is a lower energy state than the original energy state, then the photon gains energy, which results in a higher frequency. This mode is referred to as anti-Stokes Raman scattering.

The Stokes Raman scattering and anti-Stokes Raman scattering may be used to generate a spectrum. This spectrum is usually displayed having a horizontal axis corresponding to wavenumber, which is typically defined as:

$$\frac{1}{{λ}_{0}} - \frac{1}{{λ}_{1}} ,$$

where λ₀ is the wavelength of the laser and λ₁ is the wavelength of the Raman scattered light. The vertical axis is used to represent intensity.

Importantly, each molecule, when excited, produces a unique spectrum that may be used to identify that molecule. Thus, the presence of different molecules may be determined using this approach.

The percentage of Stokes-Raman scatting as compared to Rayleigh scattering is very low and highly sensitive to noise. For example, ambient light from the sun or interior lighting may alter the Raman spectrum.

Therefore, it would be advantageous if there were a system and method for reducing the amount of stray light noise that enters an optical Raman probe sensor.

### SUMMARY

A cap having a closed end and one or more openings is attached to the tip of an optical Raman probe sensor. The cap serves to block stray light noise from entering the tip of the sensor. In this way, Raman spectra may be more accurate and consistent. Further, the cap may be permanently affixed or removably attached to the sensor. In some embodiments, a reflective surface may be included on the interior surface of the closed end of the cap. This reflective surface may reflect Raman scattering light toward the tip, enhancing the received signal by a factor of 2 to 100.

According to one embodiment, a device to measure Raman scattering is disclosed. The device comprises an optical Raman probe sensor; a tube surrounding the optical Raman probe sensor, wherein light from a laser travels through the tube and through a window; and a cap, attached to the tube, comprising: a cylindrical body having one or more openings; and a closed end, wherein the cap is disposed on an end of the tube such that the light from the laser travels toward the closed end. In some embodiments, the openings comprise between 25% and 75% of the circumference of the cylindrical body. In some embodiments, the device comprises a reflective surface disposed at an interior surface of the closed end. In certain embodiments, the reflective surface comprises a mirror and the mirror may be a concave mirror. In certain embodiments, the reflective surface comprises a coating applied to the interior surface of the closed end. In some embodiments, the cap is welded to the tube. In some embodiments, the cap is constructed from a material that provides spectral blocking of wavelengths from 400 nm to 2000 nm. In certain embodiments, the cap is constructed from stainless steel, a plastic or a polymer.

According to another embodiment, a bioreactor system is disclosed. The bioreactor system comprises a bioreactor having a bioreactor bag disposed therein; and the device described above, wherein the tube is disposed within the bioreactor bag.

According to another embodiment, a device to measure Raman scattering is disclosed. The device comprises an optical Raman probe sensor; a tube comprising a tube body surrounding the optical Raman probe sensor, wherein light from a laser travels through the tube body; and a tube head affixed to the tube body, wherein the tube head comprises a window; and a cap, removably attached to the tube, comprising: a cylindrical body having one or more openings; and a closed end, wherein the cap is disposed on an end of the tube such that the light from the laser travels through the window and toward the closed end. In some embodiments, the tube head comprises external threads, and threads are disposed on the interior surface of the cylindrical body. In some embodiments, the openings comprise between 25% and 75% of the circumference of the cylindrical body. In some embodiments, the device comprises a reflective surface disposed at an interior surface of the closed end. In certain embodiments, the reflective surface comprises a mirror and the mirror may be a concave mirror. In certain embodiments, the reflective surface comprises a coating applied to the interior surface of the closed end.

According to another embodiment, a bioreactor system is disclosed. The bioreactor system comprises a bioreactor having a bioreactor bag disposed therein; and the device described above, wherein the tube body is disposed within the bioreactor bag.

### BRIEF DESCRIPTION OF THE FIGURES

For a better understanding of the present disclosure, reference is made to the accompanying drawings, which are incorporated herein by reference and in which:

FIG. 1 illustrates a bioreactor that includes a Raman probe sensor;

FIGS. 2A-2B show an exploded view of the improved Raman probe sensor and a cross-section of an assembled Raman probe sensor, respectively, according to one embodiment;

FIGS. 3A-3B show an exploded view of the improved Raman probe sensor and a cross-section of an assembled Raman probe sensor, respectively, according to a second embodiment;

FIGS. 4A-4B show the cap with two different configurations of openings;

FIG. 5 shows a reflective surface disposed at the closed end of the cap;

FIGS. 6A-6B show two graphs that illustrate the benefits of the cap in reducing stray light noise; and

FIGS. 7A-7C show three orientations of the cap on the optical Raman probe sensor.

### DETAILED DESCRIPTION

Embodiments of the present disclosure describe the system and method for reducing stray light noise in an optical Raman probe sensor.

In many applications, such as bioprocessing applications, it is important to carefully and accurately monitor the materials within the bioreactor.

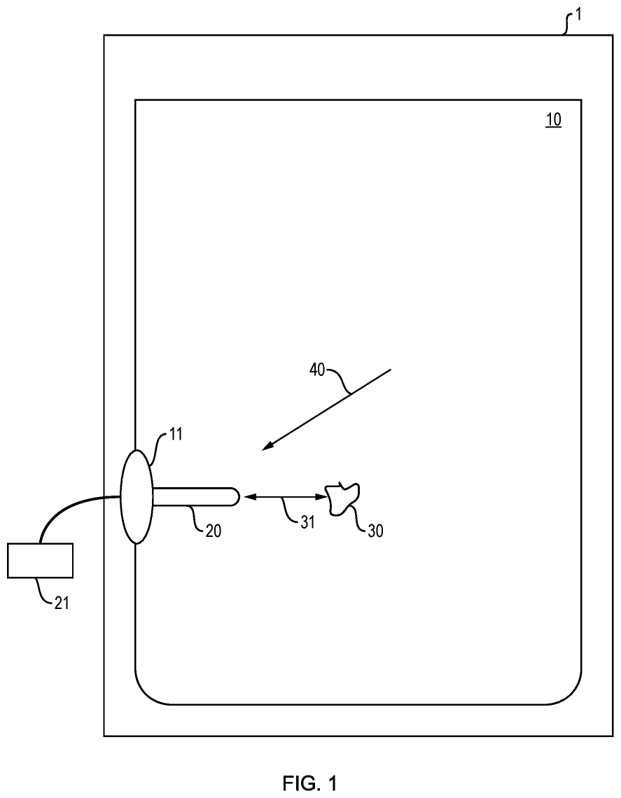

FIG. 1 shows a representative bioreactor 1. A bioreactor bag 10 is typically inserted in the bioreactor 1. The bioreactor bag 10 may have a plurality of ports to allow the introduction of various sensors, actuators, spargers or other mechanisms into the interior of the bioreactor bag 10. In this illustration, the optical Raman probe sensor 20 enters the interior of the bioreactor bag 10 through port 11. The optical Raman probe sensor 20 includes a tube with a sapphire window. The optical Raman probe sensor 20 also includes a connection thread that is compatible with bioreactors, such as PG13.5. In some embodiments, the tube of the optical Raman probe sensor 20 has a maximum external diameter of 12 mm. The optical Raman probe sensor 20 is immersed in the material contained within the bioreactor bag 10. The optical Raman probe sensor 20 projects a laser beam along an optical axis 31. The laser beam passes through the tube, the sapphire window and into the bioreactor bag 10. The optical Raman probe sensor 20 also receives scattering light from a target 30, which may be a molecule or group of molecules. The scattering light travels along a collection field of view, and enters the tip of the optical Raman probe sensor 20. The optical Raman probe sensor 20 may include an optical detector, such as a CCD or photodetector.

The optical Raman probe sensor 20 is in communication with a Raman analyzer 21, which is exterior to the bioreactor 1. The Raman analyzer 21 may include a laser, which generates a laser beam that travels through a conduit to the optical Raman probe sensor 20. This conduit may be a fiberoptic cable. The Raman analyzer 21 also includes a processing unit to interpret the output from the optical detector, which is transmitted from the probe to the Raman analyzer 21 through a conduit.

As noted above, Stokes Raman scattering occurs much less frequently than Rayleigh scattering, and is consequently very sensitive to noise. Therefore, ambient light 40 that enters the optical Raman probe sensor 20 may adversely the accuracy of the detection. This ambient light may be sunlight, moonlight, room lighting or other types of lighting. One way to address this is to reduce the amount of ambient light that is able to enter the optical Raman probe sensor 20. Specifically, the tip of the optical Raman probe sensor 20 may receive light from a wide field of view. By reducing the collection field of view, while not negatively impacting the ability to receive Stokes-Raman scattering, the noise can be at least partially eliminated.

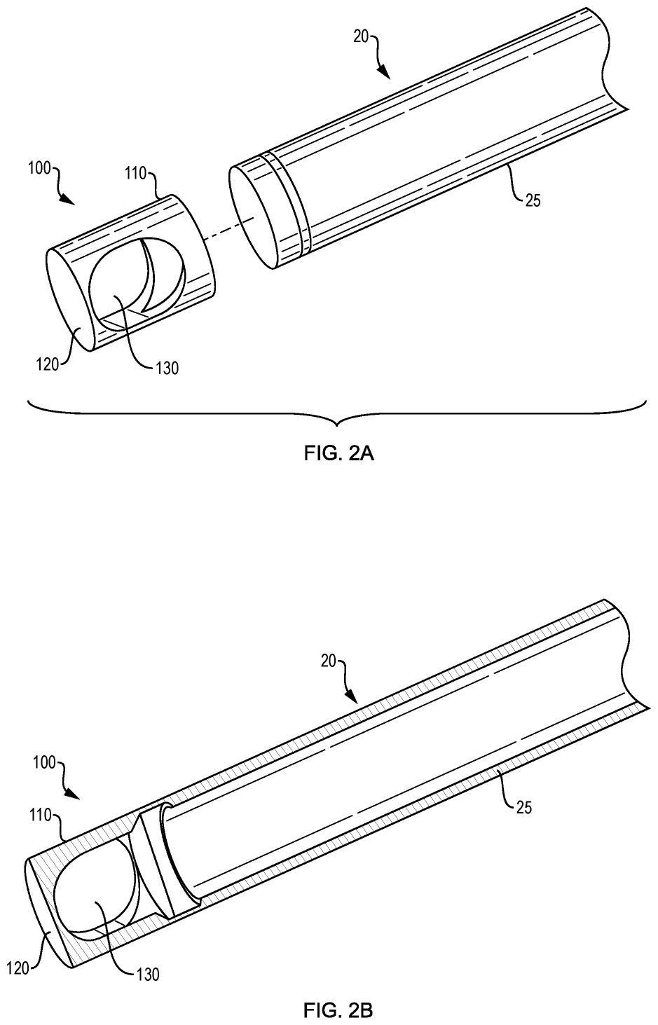

FIGS. 2A-2B show one embodiment that achieves these objectives. FIG. 2A shows an exploded view of this embodiment, while FIG. 2B shows a cross-section of the assembled sensor.

As shown in these figures, a cap 100 may be disposed over the tip of the optical Raman probe sensor 20. This cap 100 is constructed from a material that provides spectral blocking of wavelengths from 400 nm to 2000 nm. The optical Raman probe sensor 20 may be encased in a cylindrical tube. The tube 25 may be constructed from stainless steel, such as SST 316L, or other materials, such as Titane, Hastelloy, or gold. The tube 25 may have a maximum external diameter of 12 mm. A sapphire window may be disposed in the tube 25.

The cap 100 has a cylindrical body 110. The cylindrical body 110 may have a length of between 3 mm and 50 mm. In certain embodiments, the length may be less than 20 mm, such as about 10 mm. The inner diameter of the cylindrical body 110 may be slightly larger than the outer diameter of the tube 25 surrounding the optical Raman probe sensor 20 so that the cylindrical body 110 may slide over at least a portion of the tube 25 surrounding the optical Raman probe sensor 20.

The cap 100 also includes a closed end 120 disposed at the distal end of the cylindrical body 110. The distal end is the end that is opposite the end that is affixed to the tube 25. Further, the distal end is the end toward which the laser beam is directed. Additionally, the cylindrical body 110 includes one or more openings 130 disposed along the circumference of the cylindrical body 110. The openings may have a length of between 0.1 mm and 50 mm. The openings may have any desired width. In certain embodiments, the width of the openings may be as small as 0.1 mm. In other embodiments, the width of the openings may be as large as 98% of the circumference of the cylindrical body 110. In most embodiments, the width of the openings is between 25% and 75% of the circumference of the cylindrical body 110. In one specific embodiment, the total width of the openings may be equal to 50% of the circumference of the cylindrical body 110. In certain embodiments, there may be two openings 130 which are disposed on opposite sides of the cylindrical body 110. In this way, material from within the bioreactor bag 10 is able to flow through the interior of the cap 100. The openings 130 may be any suitable dimension that allows the flow of material so that the material passes through the optical axis 31.

The use of a cap 100 reduces the amount of stray light that is able to reach the tip of the optical Raman probe sensor 20. Specifically, light cannot pass through the closed end 120. Further, light cannot pass through the closed portions of the cylindrical body 110. Thus, the amount of stray light is limited to that light which is able to pass through the openings 130 and reach the tip of the optical Raman probe sensor 20.

In one embodiment, the cap 100 may be made from stainless steel and may be laser welded to the tube 25 that surrounds the optical Raman probe sensor 20. In this way, the cap 100 becomes permanently affixed to the tube 25 surrounding the optical Raman probe sensor 20.

However, in certain embodiments, it may be advantageous to remove the cap from the optical Raman probe sensor 20, such as to clean or replace.

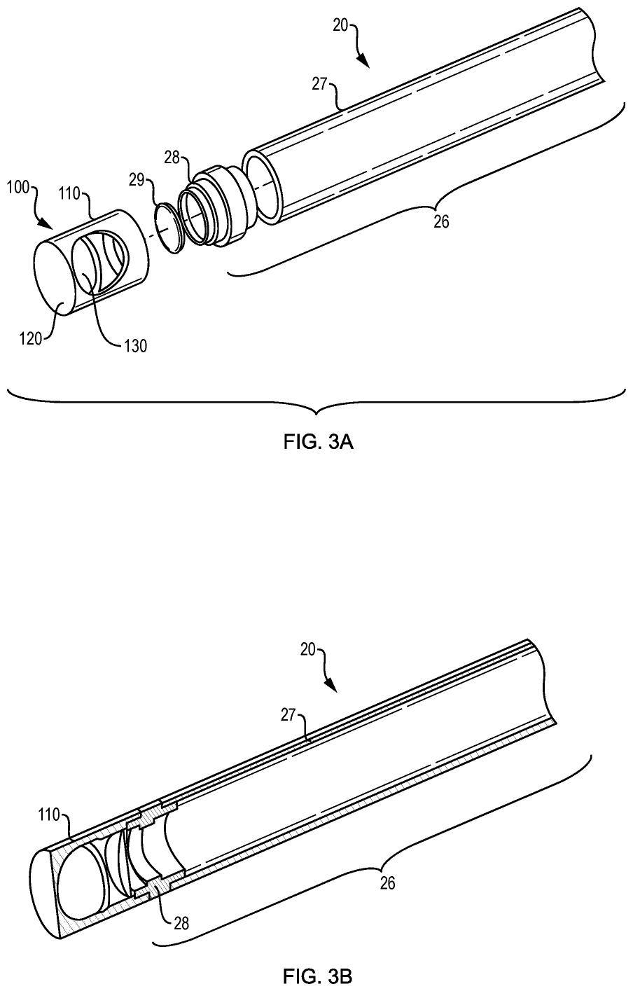

FIGS. 3A-3B shows such an embodiment. FIG. 3A shows an exploded view of this embodiment, while FIG. 3B shows a cross-section of an assembled sensor. In this embodiment, the optical Raman probe sensor 20 is enclosed in a tube 26 that comprises two parts; a tube body 27 and a tube head 28. The tube body 27 may be similar in composition to the tube 25 described with respect to FIG. 2A-2B and comprises a hollow tube. The tube head 28 is affixed to the tube body 27, such as by welding. The tube head 28 comprises a sapphire window and an optical lens 29. Further, the exterior surface of the tube head 28 includes a thread to receive the cap 100.

The ability to remove the cap 100 for a cleaning process may require a removable concept. Also, in some embodiments, the tube head 28 has threads on its exterior surface near the distal end.

In this embodiment, the cap 100 is similar to that described above but also includes a thread on the interior surface of the cylindrical body 110. In operation, the cap 100 is screwed onto the tube head 28. The cap 100 may be removed for easier cleaning. Further, in some embodiments, the cap 100 may be considered a disposable component such that a new cap 100 is installed on the tube head 28 before each use. Further, this configuration allows the possibility to choose a cap design according to the application without changing other portions of the optical Raman probe sensor 20.

In these embodiments, the length of the cap 100 may be a design decision. For example, the cap 100 may be designed such that the distance from the tip of the optical Raman probe sensor 20 to the closed end 120 is between 1 and 10 cm, although other dimensions are also possible.

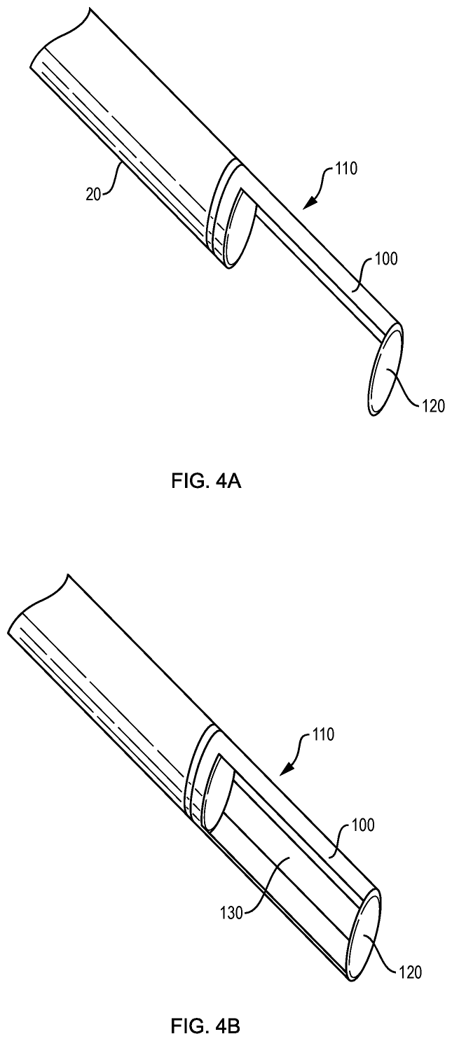

FIGS. 2A-2B and 3A-3B show the openings 130 as being two circular apertures. However, the disclosure is not limited to this embodiment. Rather, the openings may be circular, oval, rectangular or any other shape. For example, FIG. 4B shows the openings 130 as being rectangular in shape.

Further, the number of openings 130 may vary. In some embodiments, there may be more than 2 openings. In another embodiment, there is only a single opening, such as is shown in FIG. 4A. In this embodiment, the opening 130 may occupy more than 180° of the surface of the cylindrical body 110. In fact, the opening 130 may occupy any portion of the circumference of the cylindrical body 110 less than 360°. The remaining portion of the cylindrical body 110 is used to hold the closed end 120.

Further, as noted above, the size of the openings 130 may vary. For example, if there are N openings, the area occupied by these openings must occupy less than 360° of the circumference of the cylindrical body 110. Thus, if the openings 130 are of equal size, each opening must occupy less than 360°/N of the circumference of the cylindrical body 110.

The number, shape and size of the openings 130 may be varied based on the application to modulate the flow of material into the interior of the cap 100. Further, the number, shape and size of the openings 130 may also represent a tradeoff between minimizing stray light noise and allowing sufficient material flow through the interior of the cap 100.

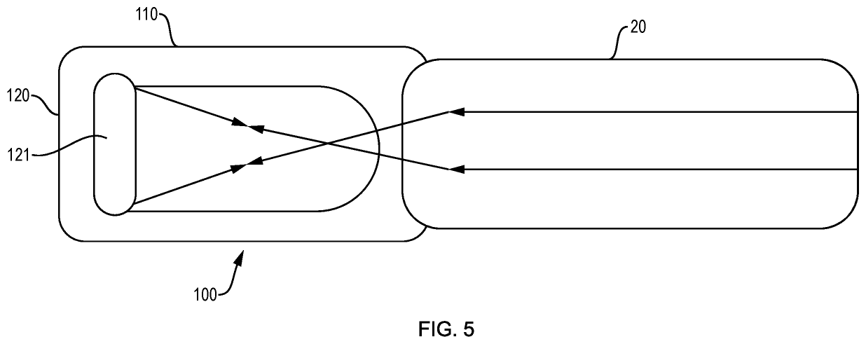

The closed end 120 may also be used to improve the sensitivity of the optical Raman probe sensor 20. FIG. 5 shows an embodiment where the closed end 120 is used to reflect more of the Stokes-Raman scattering toward the tip of the optical Raman probe sensor 20. In certain embodiments, a reflective surface 121 may be disposed on the interior surface of the closed end 120. In some embodiments, the reflective surface 121 is a treatment or coating applied directly to the interior surface of the closed end 120. In another embodiment, the reflective surface 121 is a mirror affixed to the interior surface of the closed end 120. In certain embodiments, the reflective surface 121 may be flat or planar. In other embodiments, the reflective surface 121 may be concave to direct the scattered light toward the tip of the optical Raman probe sensor 20. This reflective surface may enhance the Raman scattering signal by a factor of more than 2, such as between 2 and 100.

While the disclosure noted that the cap 100 may be made from stainless steel, other materials may also be used. For example, the cap 100 may be plastic or polymer based. In this case, the cap 100 may be secured to the tube by overmolding, thermal sealing, crimping or seaming. The cap may also be constructed from Hastelloy alloys or another material having a low Raman signature.

Further, the cap 100 is designed to avoid any rough surfaces or edges to avoid the accumulation of medium, dust, components and to facilitate cleaning with an ultrasonic bath or other cleaning procedure.

The embodiments described above in the present application may have many advantages. First, the closed end of the cap 100 ensures that the laser beam does not exit the bioreactor. Thus, the laser beam can be completely contained by the cap 100. This reduces the laser exposure of a user that may be located along the optical axis 31.

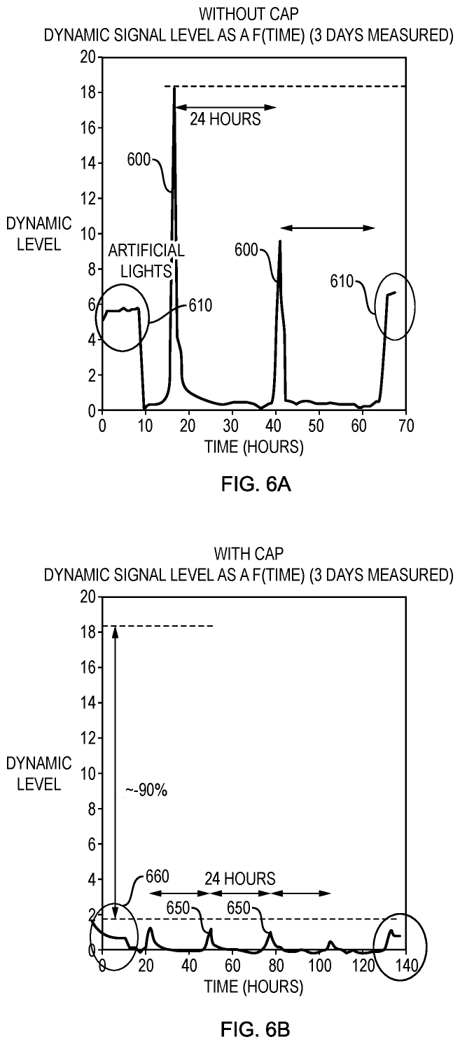

Additionally, in many applications, the optical Raman probe sensor 20 is disposed in glass bioreactors or in plastic bioreactor bags that are not fully opaque to ambient light. The cap 100 allows a Raman measurement with a drastic reduction of the impact from the interference caused by ambient light. As an example, FIGS. 6A-6B show the results of one experiment that was performed to demonstrate this benefit. In these graphs, the vertical axis represents the sum of all Raman frequency intensities measured by the optical Raman probe sensor 20. The horizontal axis represents time. The graph in FIG. 6A shows a Raman spectrum acquired using a traditional optical Raman probe sensor, where the bioreactor is disposed in a room having windows. Note that every 24 hours, there is a sharp peak 600 in the intensity of the Raman spectrum. This may be caused by sunlight, which is greatest when the position of the sun is best aligned with the collection field of view. Further, also note lower plateaus 610 are also present. These lower plateaus 610 correspond with times during which the room was illuminated.

FIG. 6B shows the results when the same optical Raman probe sensor is now used with the caps 100 described above. Note that the peaks 650 is now significantly lower than those in FIG. 6A. In fact, there is a 90% reduction in intensity of these peaks. Additionally, the plateaus 660 caused by the lighting in the room are also significantly reduced.

The decrease in stray light noise allows for better consistency of the predictions delivered by the Raman analyzer 21, leading to controlled measurement tolerances.

Another advantage is the compliance with the probe diameter standard, which is 12 mm, and compatibility with many standard connectors, such as PG13.5

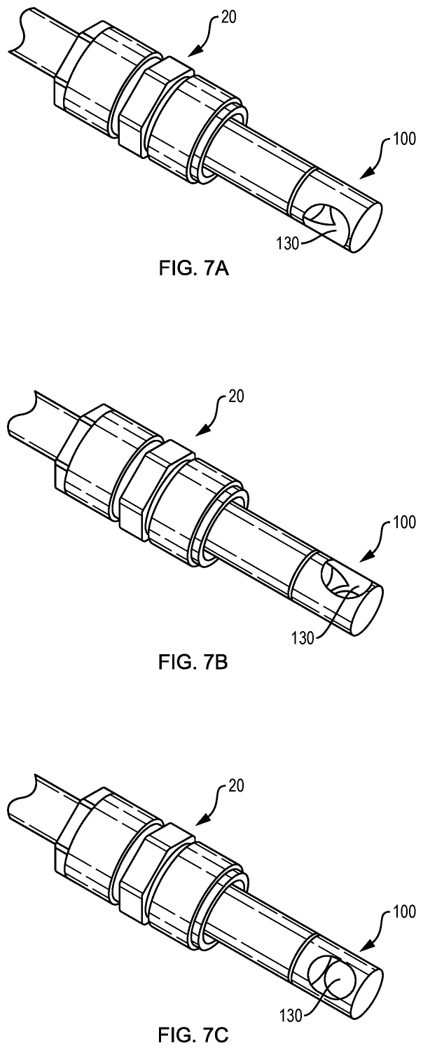

Additionally, the cap 100 may be oriented to minimize the amount of stray light noise that reaches the tip of the optical Raman probe sensor 20. For example, FIGS. 7A-7C show three different configurations where the openings 130 are oriented horizontally, vertically and at a 45° angle, respectively. Importantly, the orientation angle may be adjusted while ensuring that requisite tightness with the bioreactor connector. Further, the orientation angle may be adjusted based on the specific application or the laser safety. Additionally, the orientation angle may be adjusted based on the primary direction of the stray light. In other words, if stray light predominantly arrives from a certain direction, the cap 100 may be oriented such that the openings 130 are not aligned with this direction, thereby reducing the amount of stray light noise that reaches the tip of the optical Raman probe sensor 20.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A device to measure Raman scattering, comprising:
an optical Raman probe sensor;
a tube surrounding the optical Raman probe sensor, wherein light from a laser travels through the tube and through a window; and
a cap, attached to the tube, comprising:
a cylindrical body having one or more openings; and
a closed end,
wherein the cap is disposed on an end of the tube such that the light from the laser travels toward the closed end.

2. The device of claim 1, wherein the openings comprise between 25% and 75% of a circumference of the cylindrical body.

3. The device of claim 1, further comprising a reflective surface disposed at an interior surface of the closed end.

4. The device of claim 3, wherein the reflective surface comprises a mirror.

5. The device of claim 4, wherein the mirror is a concave mirror.

6. The device of claim 3, wherein the reflective surface comprises a coating applied to the interior surface of the closed end.

7. The device of claim 1, wherein the cap is welded to the tube.

8. The device of claim 1, wherein the cap is constructed from a material that provides spectral blocking of wavelengths from 400 nm to 2000 nm.

9. The device of claim 8, wherein the cap is constructed from stainless steel, a plastic or a polymer.

10. A bioreactor system, comprising:
a bioreactor having a bioreactor bag disposed therein; and
the device of claim 1, wherein the tube is disposed within the bioreactor bag.

11. A device to measure Raman scattering, comprising:
an optical Raman probe sensor;
a tube comprising:
a tube body surrounding the optical Raman probe sensor, wherein light from a laser travels through the tube body; and
a tube head affixed to the tube body, wherein the tube head comprises a window; and
a cap, removably attached to the tube, comprising:
a cylindrical body having one or more openings; and
a closed end,
wherein the cap is disposed on an end of the tube such that the light from the laser travels through the window and toward the closed end.

12. The device of claim 11, wherein the tube head comprises external threads, and threads are disposed on an interior surface of the cylindrical body.

13. The device of claim 11, wherein the openings comprise between 25% and 75% of a circumference of the cylindrical body.

14. The device of claim 11, further comprising a reflective surface disposed at an interior surface of the closed end.

15. The device of claim 14, wherein the reflective surface comprises a mirror.

16. The device of claim 15, wherein the mirror is a concave mirror.

17. The device of claim 14, wherein the reflective surface comprises a coating applied to the interior surface of the closed end.

18. A bioreactor system, comprising:
a bioreactor having a bioreactor bag disposed therein; and
the device of claim 11, wherein the tube body is disposed within the bioreactor bag.
